(19) United States
(12) Patent Application Publication
(10) Pub. No.: US 2026/0157716 A1
(43) Pub. Date: Jun. 11, 2026

(54) SPECIALIZED X-RAY HOLDER FOR PATIENTS WITH DEVELOPMENTAL DISABILITIES OR LIMITED BEHAVIORAL COOPERATION

(71) Applicant: The Regents of the University of Colorado, a body corporate, Denver, CO (US)

(72) Inventor: Chaitanya Puranik, Aurora, CO (US)

(21) Appl. No.: 19/537,663

(22) Filed: Feb. 12, 2026

Related U.S. Application Data
(63) Continuation of application No. PCT/US24/42624, filed on Aug. 16, 2024.
(60) Provisional application No. 63/533,372, filed on Aug. 18, 2023.

(51) Int. Cl. A61B 6/51; A61B 6/04

(52) U.S. Cl. CPC A61B 6/512; A61B 6/04

(57) ABSTRACT

A specialized oral sensory device having a housing for X-ray films or sensors and a method of capturing dental X-ray images for patients with developmental disabilities or limited behavioral cooperation. The device provides one or more oral sensory members. The one or more oral sensory members includes a receipt configured to receive an X-ray film or sensor. As a patient chews on the oral sensory member, a healthcare professional can capture X-ray images of the patient's teeth.

### PRIORITY INFORMATION

This nonprovisional application is a continuation of PCT International Patent Application No. PCT/US2024/042624 entitled “Specialized X-Ray Holder for Patients with Developmental Disabilities or Limited Behavioral Cooperation” with an international filing date of Aug. 16, 2024, by the same inventor, which claims priority to provisional application No. 63/533,372, entitled “Specialized X-Ray Holder for Patients with Developmental Disabilities or Limited Behavioral Cooperation,” filed Aug. 18, 2023 by the same inventor.

### BACKGROUND OF THE INVENTION

### Field of the Invention

This invention relates, generally, to medical devices. More specifically, it relates to medical devices configured to aid in the capturing of x-ray images.

### Brief Description of the Prior Art

Currently getting dental x-rays on patients with developmental disabilities is next to impossible on an outpatient basis. Healthcare providers are often forced into sedating patients to capture dental x-rays. As a result, the patients are taken to a hospital setting and sedated in order to acquire the dental X-ray images. As a result, the cost of acquiring these x-rays is substantially more expensive in comparison to patients without developmental disabilities. In addition, these patients receive less frequent x-ray imaging, which leads to missed dental carious lesions thereby impacting the patients' overall oral health. Furthermore, patients with developmental disabilities or limited cooperation have a sensory integration or processing disorder. Such patients use ‘chew toys’0 or ‘bite sticks’ to soothe them during stressful situations such as visiting unfamiliar or crowded places.

Accordingly, what is needed is a system and method that enables patients with developmental disabilities to tolerate the capturing of dental x-rays on a routine outpatient basis. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for an improved oral sensory imaging device and method of capturing dental images is now met by a new, useful, and nonobvious invention.

The present invention relates to an oral sensory imaging device designed for patients with developmental disabilities or limited behavioral cooperation. The device comprises an oral sensory member, which includes a first surface, a second surface, and sidewalls extending between the two surfaces. A plurality of sensory elements are disposed on the first surface and/or the second surface of the oral sensory member, and a receipt is disposed within the oral sensory member to receive an X-ray element.

The device further includes a main body interconnected with the oral sensory member. In some cases, an arm member extends between a main body and the oral sensory member. Moreover, the main body resides within a plane, and the oral sensory member is oriented at a predetermined angle with respect to this plane, where the angle is between approximately 35 degrees and approximately 45 degrees.

The device may also include a plurality of oral sensory members interconnected with a main body, where each oral sensory member includes a receipt configured to receive the X-ray element. In some embodiments, the device includes a pair of diametrically opposed oral sensory members secured to the main body. Furthermore, the plurality of oral sensory members can be equidistantly spaced around the perimeter of the main body. In these embodiments, the main body resides within a first plane, and each oral sensory member is oriented at a predetermined angle with respect to the first plane, typically between approximately 35 degrees and approximately 45 degrees.

Additionally, the receipt in the oral sensory member can be configured to withstand a bite force of at least 100 Newtons. The receipt may further include a pair of strain release slots disposed on opposing sides of the receipt, with each strain release slot having a height larger than the height of the receipt.

Each of the plurality of sensory elements can be an alphanumeric character, have a polygonal cross-section, and/or have a wave-like pattern. In some embodiments, each of the plurality of sensory elements has an outward extent that is approximately 1 mm or less, or in some cases, approximately 0.5 mm or less. Additionally, the oral sensory member may include an element-free area on the first surface and/or the second surface in overlying relation to the receipt.

Embodiments may further include an attachment point to secure a necklace cord to the device.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is a perspective view of an embodiment of the present invention.

FIG. 2 is an alternative perspective view of the embodiment from FIG. 1.

FIG. 3 is a plan view of the embodiment from FIG. 1.

FIG. 4 is an elevation view of the embodiment from FIG. 1.

FIG. 5 is a close-up view of an embodiment of a single oral sensory member.

FIG. 6 is a perspective view of an embodiment of the present invention.

FIG. 7 is a top view of an embodiment of the present invention.

FIG. 8 is a bottom view of an embodiment of the present invention.

FIG. 9 is side view of an embodiment of the present invention.

FIG. 10 is a perspective view of an embodiment of the present invention.

FIG. 11 is a side view of an embodiment of the present invention.

FIG. 12 is a bottom view of an embodiment of present invention.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized, and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

All numerical designations, such as measurements, efficacies, physical characteristics, forces, and other designations, including ranges, are approximations which are varied up or down by increments of 1.0 or 0.1, as appropriate. It is to be understood, even if it is not always explicitly stated that all numerical designations are preceded by the term “about.” As used herein, “about” or “approximately” refers to being within an acceptable error range for the particular value as determined by one of ordinary skill in the art, which will depend in part on how the value is measured or determined. As used herein, the term “about” refers to ±10% of the numerical; it should be understood that a numerical including an associated range with a lower boundary of greater than zero must be a non-zero numerical, and the term “about” should be understood to include only non-zero values in such scenarios.

The present invention encompasses various embodiments that incorporate one or more of the features described herein to address different aspects and applications of the oral sensory imaging device. Each of these features can be incorporated individually or in combination in each of the various embodiments to provide a comprehensive solution for enhancing the usability, effectiveness, and patient comfort of dental imaging devices, particularly for patients with developmental disabilities or limited behavioral cooperation. The specific inclusion of features such as the sensory elements, receipts for X-ray elements, main body configurations, and various structural adaptations, can be selectively implemented in different embodiments or included in each embodiment to address specific needs and use cases.

The present invention includes a specialized oral sensory device having a housing for X-ray film and/or X-ray sensors and a method of capturing dental X-ray images for patients with developmental disabilities during clinical or radiographic dental exam on an outpatient or inpatient basis. The device provides one or more oral sensory members/pads that contain receipts for housing the X-ray film or sensor and is configured to withstand repeated biting or chewing. As a patient chews on the oral sensory member, a healthcare professional can capture X-ray images of the patient's teeth.

The device, or at least the oral sensory members of the device, are comprised of biocompatible/food grade material to ensure that the device is safe for the patient. In addition, at least the oral sensory members are comprised of a material with sufficient strength to withstand the typical bite force of humans. In some embodiments, the composition of the device is also capable of withstanding cleaning through conventional autoclaves for embodiments that are intended to remain with the healthcare provider.

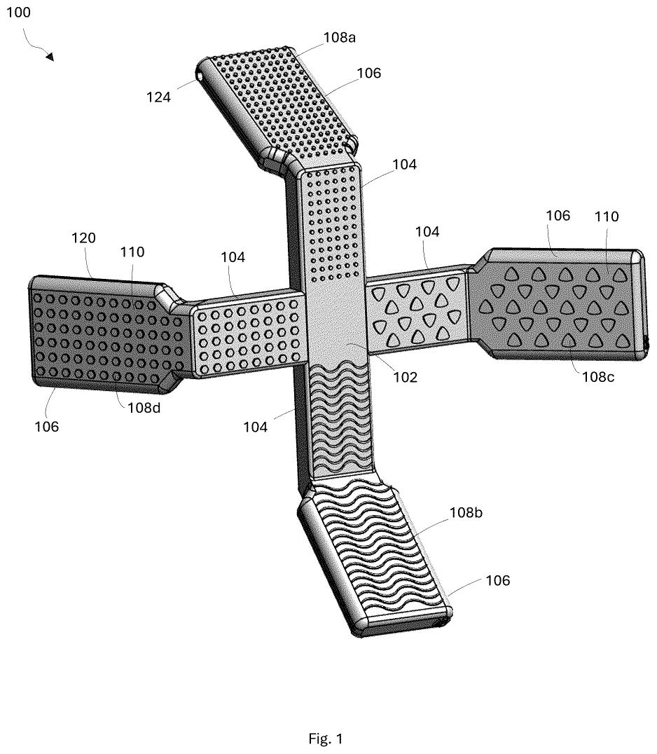

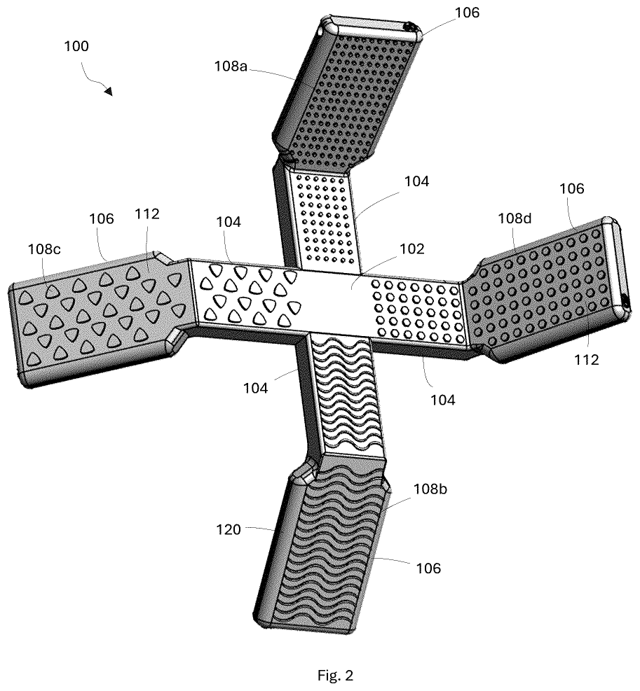

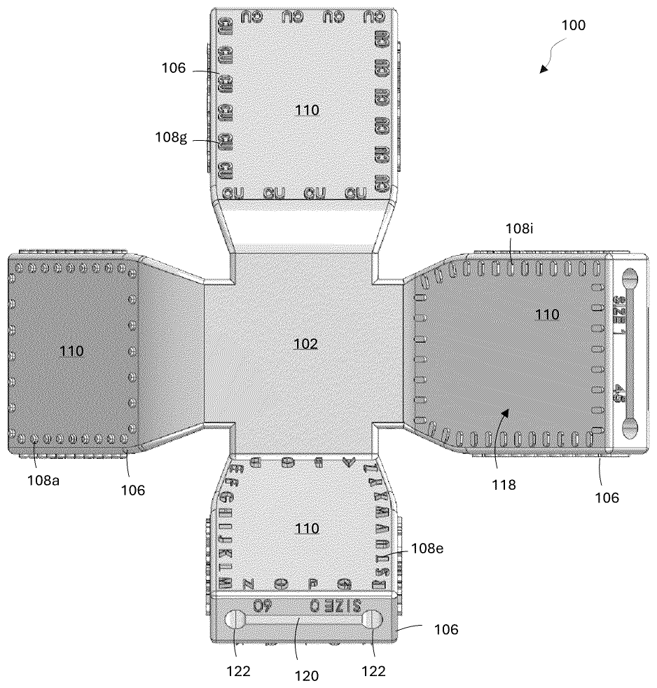

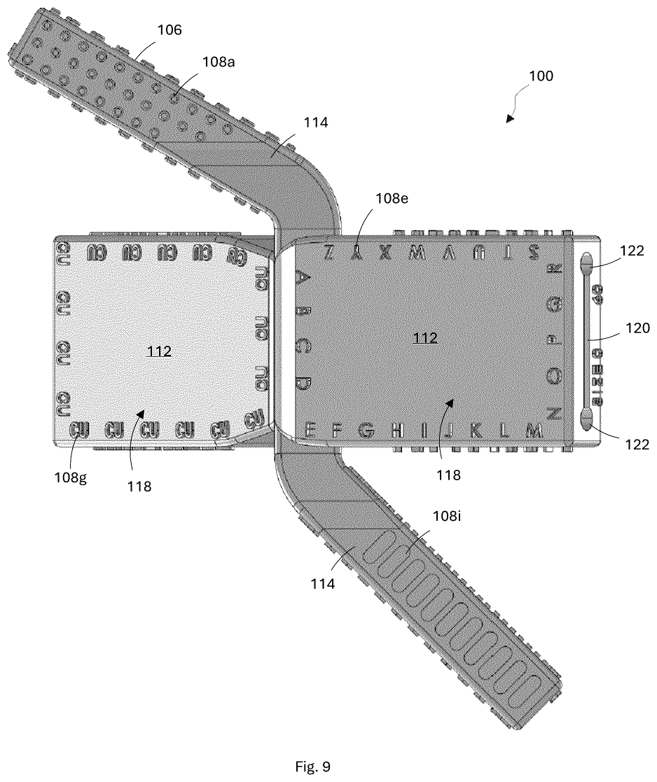

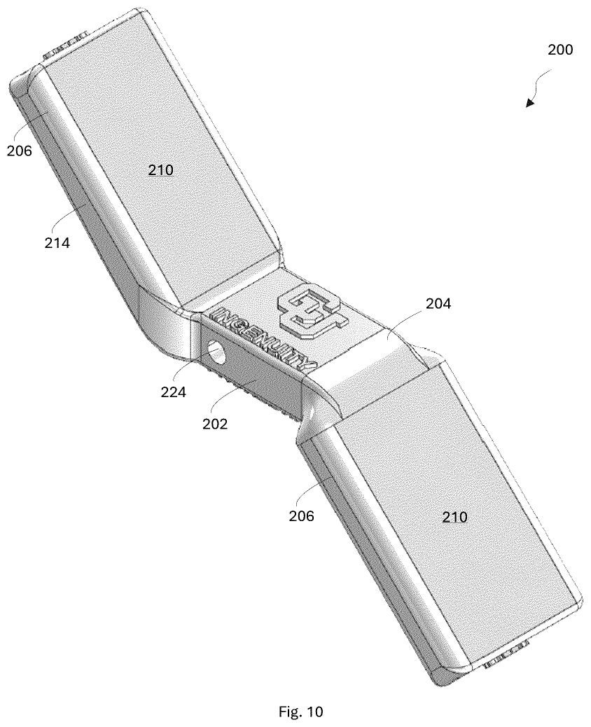

Referring now to FIGS. 1-9, embodiments of oral sensory imaging device 100 include main body 102 with a multitude of arm members 104 each extending to oral sensory members 106. While the embodiments depicted in FIGS. 1-9 include four arms and four oral sensory members 106, alternative embodiments can include more (five or more) or fewer (2 or less), such as embodiment 200 depicted in FIGS. 10-12, which includes two arms 204 and two oral sensory members 206. Moreover, in some embodiments, oral sensory members 106 are part of or effectively part of main body 102 of the device.

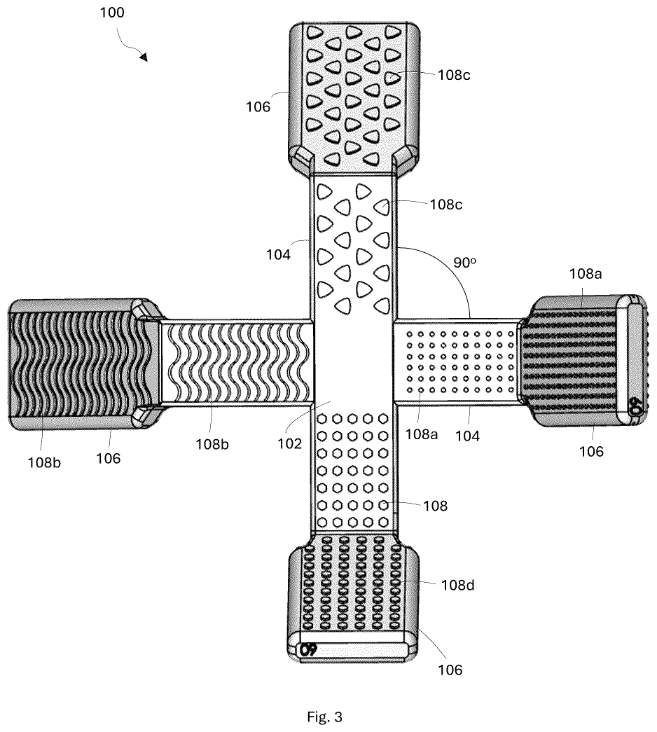

Main body 102 serves as a central hub from which arms 104 and/or oral sensory members 106 extend. Arms 104 may be equidistantly spaced around main body 102 in an effort to create ample spacing so that oral sensory members 106 can reside within a patient's mouth without other arms 104 and/or oral sensory members 106 contacting the patient's face. In some embodiments, arms 104 are circumferentially spaced at approximately 90-degree increments as shown in FIG. 3.

Likewise, each arm 104 has a sufficient length to ensure that oral sensory member 106 can reside within a patient's mouth without the other arms 104 and oral sensory members 106 contacting the patient's face. In some cases, the length of each arm 104 is between approximately 0.75 inches and approximately 2 inches.

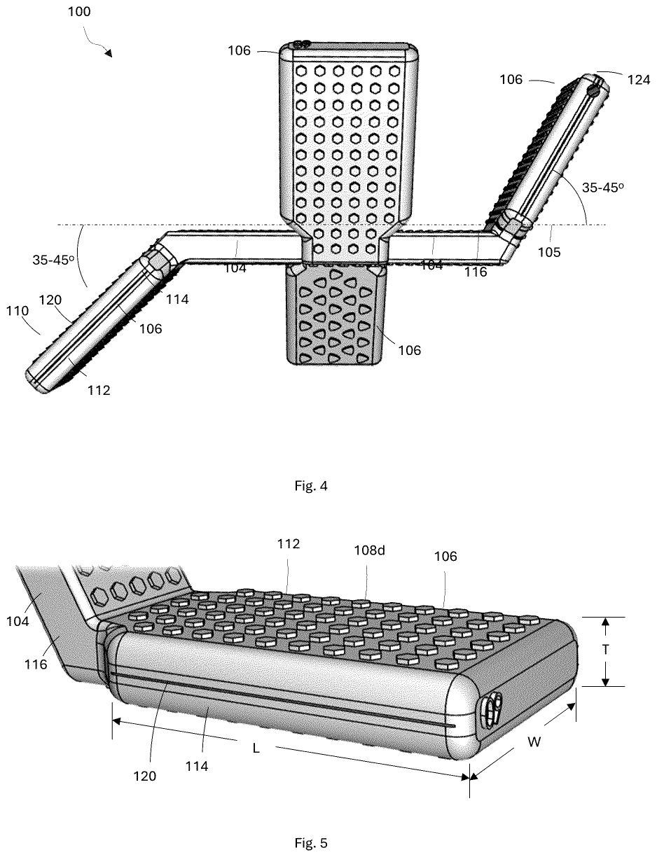

As will be explained in subsequent paragraphs, oral sensory members 106 are intended to house the X-ray element (e.g., an X-ray film or sensor). Thus, oral sensory members 106 have specific orientations and dimensions to achieve this functionality. As best shown in FIG. 4, some embodiments include oral sensory members 106 extending out of plane relative to plane 105 in which arms 104 and/or main body 102 reside. In some embodiments, the angle of oral sensory members 106 is between 35 and 45 degrees relative to plane 105. The angle helps to place oral sensory members 106, and in turn, the X-ray element into an orientation within the patient's mouth that increases the odds of capturing diagnostic images of the patient's teeth.

As illustrated in FIG. 5, each oral sensory member 106 has a predetermined length ‘L’. The length of one oral sensory member 106 may vary relative to another oral sensory member 106 to provide differing extents into a patient's mouth, which could be useful in capturing images of different teeth. In some embodiments, the length ‘L’ of oral sensory members 106 is between approximately 1 inches and approximately 2.5 inches.

Each oral sensory member 106 also includes a predetermined width ‘W’ to fit into the mouth of a patient and also adequately house an X-ray film. The width may vary depending on the age of the patient and the size of the patient's mouth. The width may also vary from one oral sensory member 106 relative to another oral sensory member 106, which could be useful in capturing images of different teeth. In some embodiments, the width may be between approximately 0.3 inches (0.75 cm) and approximately 0.8 inches (2 cm).

Each oral sensory member 106 also includes a predetermined thickness T to fit into the mouth of a patient and also adequately house at least a traditional X-ray film commonly used in dental practices (e.g., Size 0:22×35 mm, Size 1:24×40 mm, and Size 2:31×41 mm). The thickness may vary depending on the age of the patient and the size of the patient's mouth. For example, the thickness may be between approximately 0.2 inches (0.5 cm) to approximately 0.6 inches (1.5 cm).

Furthermore, the oral sensory members 106 can have alternative shapes in comparison to the depicted embodiment. Rather than a rectangular shape, the oral sensory members 106 can have generally any shape known in the art so long as the oral sensory member 106 includes a receipt for housing the X-ray element and can fit comfortably within a patient's mouth. In addition, multiple oral sensory members 106 on a single device can have alternative shapes relative to each other.

Patients with developmental disabilities may have a concurrent sensory integration or processing disorder. Hence, the patients may have special chew-rings or toys with sensory surfaces for desensitization. The patients can bite on the chew rings or toys during stress-inducing day-to-day functioning such as going to crowded places such as a grocery store. Similarly, oral sensory members 106 of the device include tactile surfaces having sensory elements 108 of various sizes, designs, and configurations to provide a sensory surface that is highly desirable for patients with developmental disabilities. Sensory elements 108 are also comprised of a resilient, biocompatible material (e.g., silicon) similar to the materials used in sensory chew-rings or toys.

While an oral sensory member 106 can have one or more smooth surfaces, each oral sensory member 106 can also have one or more projections or sensory elements 108 extending from one or more surfaces creating a sensory surface that might be preferred by patients with developmental disabilities and sensory integration or processing disorders. Non-limiting examples of sensory elements 108 include a series of cylindrical projections 108a, wave-like projections 108b, triangular projections 108c, and hexagonal extensions 108d (see FIGS. 1-4). Other shapes may be used as projections as well including but not limited to alphanumeric characters (e.g., letters 108e, numbers 108f, acronyms 108g, wording 108h, etc.), ovals 108i, and spherical elements (not depicted).

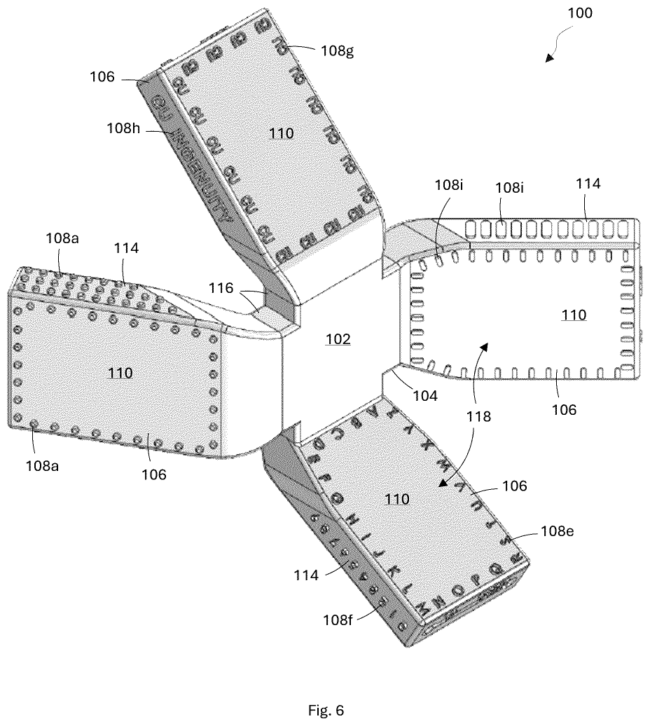

Sensory elements 108 may be equidistantly spaced, disposed on one or more surfaces in a discernible pattern, or disposed in a seemingly random pattern. In addition, sensory elements 108 can extend from first face 110, second face 112, or both faces of one or more oral sensory members 106. Sensory elements 108 could also extend outwardly from sidewalls 114 extending between the two faces (see e.g., FIG. 6) and/or the various surfaces and/or sidewalls 116 of arms 104 extending between main body 102 and oral sensory members 106.

Sensory elements 108 extend outwardly from their respective surface a predetermined distance. In some embodiments, sensory elements 108 extend outwardly approximately 1 mm or less. In some embodiments, sensory elements 108 extend outwardly approximately 0.5 mm or less. It was determined that some shapes of the silicon-based sensory elements that extended more than 1 mm produced artifacts on the imaging. With some shapes, an extension of 0.5 mm was deemed adequate for eliminating X-ray artifacts.

However, some patients prefer sensory elements 108 that extend outwardly more than 0.5 mm. Thus, as depicted in FIGS. 6-9, some embodiments include element-free areas 118 on oral sensory members 106 to eliminate X-ray artifacts. Element-free areas 118 can be located on first face 110, second face 112, or both faces. Moreover, element-free areas 118 are located in overlying relation to the receipts that house X-ray elements. For that reason, element-free areas 118 can have sizes that correspond to the size of each X-ray element intended to be used with specific oral sensory members 106 on which the element-free areas 118 are located. For example, element-free areas 118 may have a size that is approximately equal to a size 0 X-ray film (22×35 mm), a size 1 X-ray film (24×40 mm), a size 3 X-ray film (31×41 mm), etc.

As best shown in FIGS. 4-5 and 7, oral sensory members 106 can include receipts 120 for housing X-ray films or X-ray sensors to capture X-ray images. In some embodiments, receipts 120 may reside in only one or a subset of all of oral sensory members 106. Alternatively, receipts 120 may reside in each oral sensory member 106. Some embodiments may also or alternatively include receipts 120 disposed within arms 104 as well.

Each receipt 120 has a shape and size sufficient to allow for the insertion and retention of an X-ray element and its protective packaging. Some embodiments include receipts 120 of specific sizes to accommodate specifically sized X-ray elements. For example, receipts 120 may have a size that is approximately equal to or larger than a size 0 X-ray film (22×35 mm), a size 1 X-ray film (24×40 mm), a size 3 X-ray film (31×41 mm), a size 4 X-ray film (27×54 mm), and/or a size 5 X-ray film (57×76 mm). In some embodiments, receipts 120 have a length between approximately 1.25 inches and approximately 1.75 inches, and/or a width between approximately 0.3 inches and approximately 0.8 inches. In some embodiments, the height of receipt 120 is between approximately 0.75 inches and approximately 1.25 inches.

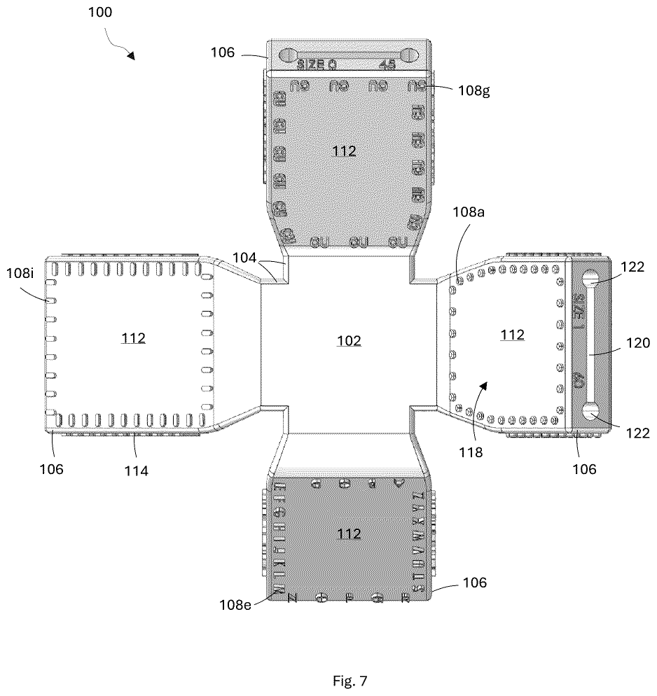

As best exemplified in FIGS. 7-9 embodiments of receipt 120 also include strain release slots 122. Strain release slots 122 help reduce the required force to remove an X-ray element from receipt 120. In some embodiments, strain release slots 122 have a cross-sectional area of approximately 0.05-0.15 inches.

For similar reasons, embodiments of receipt 120 also have a predetermined clearance with respect to the height and width of the receipt 120 relative to the height and width of the X-ray element and its packaging. The height clearance is between approximately 0.05 to 0.4 inches depending on the X-ray sizes. The width clearance is between approximately 0.8 to 1.4 inches.

Receipts 120 are further configured to withstand the bite force of the patients. In some embodiments, the receipts 120 are configured to withstand at least 200 Newtons of force, and in some cases at least 100 Newtons of force. To do so, embodiments include a sufficient amount of silicon between the outside of strain release slots 122 and the outside edge of the device to ensure that the device does not split open or break. The width of the silicon residing between the outside of strain release slots 122 and the outside edge of the device is between approximately 0.03 inches and approximately 0.15 inches. Alternatively, or additionally, receipts 120 may include additional structural supports or may be comprised of a more rigid material.

Receipts 120 may further include closures to seal the X-ray elements within receipts 120 and keep saliva out of receipts 120. The closures may be simple press-fit lids or other closure mechanisms known in the art.

As previously noted, some embodiments are configured to receive and retain X-ray sensors. Some of these sensors include electrical wiring. Thus, some embodiments of the device include wiring passages or channels (not depicted). The wiring passages may direct the wire out of the device through the oral sensory member 106 or may pass through the oral sensory member 106, the corresponding arm section 104, and exit the device through the main body 102 to move the exit aperture to a location away from the patient's mouth.

Some embodiments of the device are intended to remain with the patient following an initial visit. Therefore, some embodiments include a necklace cord to allow the patient to carry the device around his/her neck or ensure that the device is not accidentally dropped. However, embodiments intended to remain at the healthcare facility could also include a necklace cord. Such embodiments may further include a designated attachment point (e.g., through hole 124) for the necklace cord and may include a means for detaching the necklace cord at attachment point 124 or on the necklace cord itself. Attachment point 124 may be on the main body 102 or on one of the oral sensory members 106.

Referring now to FIGS. 10-12, embodiment 200 of the oral sensory imaging device includes main body 202 with two arm members 204 extending to oral sensory members 206. This embodiment is designed to provide a simpler structure while maintaining functionality for effective dental imaging. Main body 202 serves as a central hub from which arm members 204 extend in a diametrically opposed relation. Each arm member 204 is connected to oral sensory member 206 that fits comfortably within a patient's mouth.

Like embodiment 100, oral sensory members 206 have specific orientations and dimensions to locate the X-ray elements within certain areas in a patient's mouth. Oral sensory members 206 in embodiment 200 extend out of plane relative to a plane in which arm members 204 and/or main body 202 reside. The angle of the oral sensory members 206 is between 35 and 45 degrees relative to said plane, helping to place the oral sensory members 206, and in turn, the X-ray element into an optimal orientation within the patient's mouth to capture diagnostic images of the teeth.

Each oral sensory member 206 also has similar sizes and dimension to those described in reference to oral sensory members 106. Likewise, oral sensory members 206 also include receipts 220 similar to receipts 120 and receipts 220 can have the same dimensions, orientations, and features as those described in reference to receipts 120. For embodiments configured to retain X-ray sensors with electrical wiring, embodiment 200 can also include wiring passages such as those described in relation to embodiment 100.

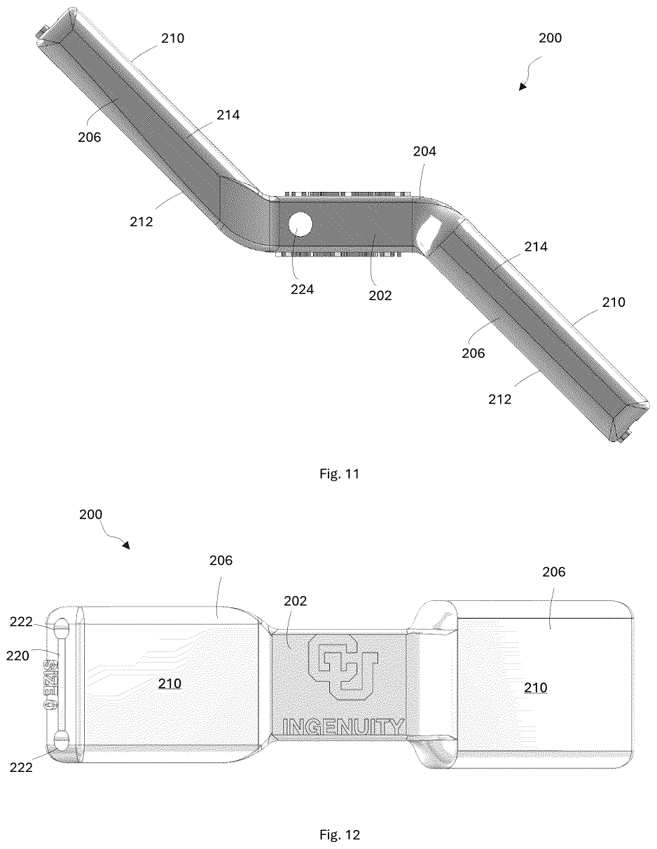

As depicted in FIGS. 10 and 12, oral sensory members 206 lack sensory elements. While some embodiments may not include sensory elements, other embodiments may include any of the various sensory elements 108 along with their respective features, design, orientation, location, etc. as described in reference to embodiment 100.

Embodiment 200 is also configured to include a necklace cord attachable via attachment point 224 on the main body 202 or alternatively on arms 204 or oral sensory members 206.

As best depicted in FIGS. 10 and 12, main body 202 (and 102) may also serve as a surface for indicia such as advertising and marketing information. Similarly oral sensory members 206 (and 106) may also include sizing indicia to indicate the size of the X-ray element intended to fit within slots 220 (and 120).

The present invention further includes a method of capturing X-rays through the use of the various embodiments of the device described above. The method can also include constructing the device based on a particular patient's preferred projection shape and/or preferred design of the oral sensory member of the device. For example, some patients may prefer cylindrical projections over a wave-like pattern, and the device can be manufactured based on that preference.

Accordingly, some embodiments of the method include receiving information containing the patient's preferred overall device shape; the size of the device; the number of oral sensory members; the size of the oral sensory members; the shape of the oral sensory members; and/or the number, shape, size and spacing of sensory elements. Responsive to acquiring this information, the method includes designing such a device for the patient.

The method further includes manufacturing the custom device with the patient's preferences. The manufacturing step can be performed using additive manufacturing, also known as 3D printing. Once the device is manufactured, the X-ray film or sensor is inserted into one of the receipts and the device is provided to the patient. A healthcare professional can then acquire the X-ray images while the patient chews on the device. The customized device could also be sent home with the patient after the X-rays are captured or sent prior to visiting the healthcare facility to allow the patient to become more acquainted/acclimatized/desensitized with the device.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. An oral sensory imaging device for patients with developmental disabilities or limited behavioral cooperation, comprising an oral sensory member, the oral sensory member further including:
a. a first surface, a second surface, and sidewalls extending between the first surface and the second surface;
b. a plurality of sensory elements disposed about the first surface, the second surface, or both of the oral sensory member; and
c. a receipt disposed within the oral sensory member, wherein the receipt is configured to receive an X-ray element.

2. The device of claim 1, further comprising a main body interconnected with the oral sensory member, the main body residing within a plane.

3. The device of claim 1, wherein the receipt in the oral sensory member is configured to withstand a bite force of at least 100 Newtons.

4. The device of claim 1, further including a plurality of oral sensory members interconnected with a main body, wherein each oral sensory member includes a receipt configured to receive the X-ray element.

5. The device of claim 1, wherein each of the plurality of sensory elements has an outward extent that is approximately 1 mm or less.

6. The device of claim 1, wherein each of the plurality of sensory elements is an alphanumeric character.

7. The device of claim 1, wherein the oral sensory member includes an element-free area on the first surface and/or the second surface in overlying relation to the receipt.

8. The device of claim 1, further comprising a pair of diametrically opposed oral sensory members secured to a main body.

9. The device of claim 1, further comprising a pair of strain release slots disposed on opposing sides of the receipt, wherein each strain release slot has a height larger than a height of the receipt.

10. The device of claim 1, further including an attachment point configured to secure a necklace cord to the device.

11. An oral sensory imaging device for patients with developmental disabilities or limited behavioral cooperation, comprising:
a. a main body
b. a plurality of oral sensory members equidistantly disposed about the main body, each oral sensory member further including:
i. a first surface, a second surface, and sidewalls extending between the first surface and the second surface
ii. a plurality of sensory elements disposed about the first surface, the second surface, or both of the oral sensory member; and
iii. a receipt disposed within the oral sensory member, wherein the receipt is configured to receive an X-ray element.

12. The device of claim 11, wherein the receipt is configured to withstand a bite force of at least 100 newtons.

13. The device of claim 11, wherein each of the plurality of sensory elements has an outward extent of 0.5 millimeters or less.

14. The device of claim 11, wherein the oral sensory member includes an element-free area on the first surface and/or the second surface in overlying relation to the receipt.

15. The device of claim 11, wherein each of the plurality of sensory elements has an outward extent that is approximately 1 mm or less.

16. The device of claim 15, wherein the main body resides within a plane and the oral sensory member is oriented at an angle between approximately 35 degrees and approximately 45 degrees relative to the plane.

17. An oral sensory imaging device for patients with developmental disabilities or limited behavioral cooperation, comprising:
a. an oral sensory member, the oral sensory member further including:
i. a first surface, a second surface, and sidewalls extending between the first surface and the second surface;
ii. a plurality of sensory elements disposed the first surface and/or the second surface of the oral sensory member, wherein each of the plurality of sensory elements has an outward extent that is approximately 1 mm or less;
iii. and a receipt disposed within the oral sensory member, wherein the receipt is configured to receive an X-ray element.

18. The device of claim 17, wherein each of the sensory member includes an element-free area on the first surface, the second surface, or both in overlying relation to the receipt.

19. The device of claim 17, further comprising an attachment point configured to secure a cord or necklace cord to the device.

20. The device of claim 17, further including a pair of strain release slots disposed on opposing sides of the receipt, wherein each strain release slot has a height larger than a height of the receipt.
